(19) United States
(12) Patent Application Publication
(10) Pub. No.: US 2025/0275944 A1
(43) Pub. Date: Sep. 4, 2025

(54) RELATIVE UNDERSUPPLY OF AN AMYLOID BETA AGGREGATION INHIBITOR FOR IMPROVED DETOXIFYING EFFECTS

(71) Applicant: Galimedix Therapeutics Inc, Kensington, MD (US)

(72) Inventors: Hermann Kurt RUSS,, Dubai (AE); Gerhard RAMMES, Wielenbach (DE)

(73) Assignee: Galimedix Therapeutics Inc, Kensington, MD (US)

(21) Appl. No.: 18/862,559

(22) PCT Filed: May 3, 2023
(86) PCT No.: PCT/IB2023/054578

§ 371 Date: Nov. 4, 2024

Related U.S. Application Data
(60) Provisional application No. 63/338,037, filed on May 4, 2022.

(51) Int. Cl. A61K 31/4045; A61P 25/28

(52) U.S. Cl. CPC A61K 31/4045; A61P 25/28

(57) ABSTRACT

Provided herein a method for treating or preventing a protein misfolding and deposition disease in a subject, by administering to the subject a low dose amount of compound (1). Provided herein is also a method for reversing or preventing the toxic effect of the misfolded and aggregated protein amyloid beta, by using compound (1) in a relative undersupply (reversed stoichiometric ratio) to achieve stronger detoxifying effects. Also provided dosages and administration thereof.

### BACKGROUND OF THE INVENTION

The majority of protein molecules must fold into defined three-dimensional structures to acquire functional activity. However, protein chains can adopt a multitude of conformational states, and their biologically active conformation is often only marginally stable. Proteins in conformational stages non-supportive of their physiological function are called misfolded. Aggregation and accumulation of misfolded proteins can cause disease, a phenomenon that increases dramatically with age. The mechanistic explanation for this correlation is that as humans age (or as a result of mutations), the delicate balance of the synthesis, folding, and degradation of proteins can be perturbed, resulting in the production and accumulation of misfolded proteins that form aggregates and induce pathologies. Protein aggregation diseases are not exclusive to the central nervous system; they can also appear in peripheral tissues.

One of the most common protein misfolding diseases is Alzheimer's disease, which is associated with the misfolding of the protein amyloid beta. Misfolded amyloid beta shows the tendency to aggregate and form oligomeric species, which are known to be highly toxic to neuronal cells causing neurodegeneration.

This amyloid beta-associated pathology isn't limited to the different forms of Alzheimer's disease, it is rather found also in type II diabetes mellitus, serum amyloid A disease (SAA amyloidosis), hereditary Icelandic syndrome, multiple myeloma, medullary carcinoma, aortic amyloidosis, cardiac amyloidosis, insulin injection amyloidosis, prion-systematic amyloidosis, chronic inflammation amyloidosis, senile systemic amyloidosis, pituitary gland amyloidosis, hereditary renal amyloidosis, familial British dementia, Finnish hereditary amyloidosis, familial non-neuropathic amyloidosis, amyloid beta-related ocular diseases, particularly glaucoma and age-related macular degeneration, and others.

The formation of pathological aggregates of misfolded proteins has been identified as promising target for new treatment approaches with the potential to slow down or even stop the progression of the neurodegenerative process. Just recently it turned out that particularly the soluble toxic amyloid beta oligomers are a promising drug target for Alzheimer's disease with first new treatments under successful clinical development and with two recently approved drug for Alzheimer's disease (aducanumab and lecanemab) approved for treatment.

Despite these first successful new drug developments targeting toxic amyloid beta oligomers and protofibrils using antibody-based infusions, their beneficial effects on the progression of Alzheimer's disease is still rather limited and there remains a high demand for more efficient, better tolerable, easier to administer, and cheaper to produce drugs.

### SUMMARY OF THE INVENTION

In some aspects, disclosed herein is a method of treating or preventing a protein misfolding and deposition disease in a subject in need thereof, the method comprising administering to the subject a low dose of compound (1).

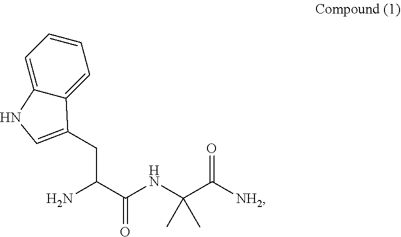 [SMILES: CC(C)(NC(=O)C(N)Cc1c[nH]c2ccccc12)C(N)=O]

thereby treating or preventing the protein misfolding and deposition disease in said subject.

In some related aspects, the low dose administration results in a concentration of less than 50 nM at the site of action in the brain. In some related aspects, the low dose administration results in a concentration of 10 nM at site of action. In some related aspects, the low dose administration results in a concentration of 3 nM at site of action. In some related aspects, the low dose administration results in a concentration of 1 nM at site of action.

In some related aspects, the protein misfolding and deposition disease is selected from diseases associated with amyloid beta pathology, namely Alzheimer's disease (AD), early onset Alzheimer's disease, late onset Alzheimer's disease, pre-symptomatic Alzheimer's disease, type II diabetes mellitus, serum amyloid A disease (SAA amyloidosis), hereditary Icelandic syndrome, multiple myeloma, medullary carcinoma, aortic amyloidosis, cardiac amyloidosis, insulin injection amyloidosis, prion-systematic amyloidosis, chronic inflammation amyloidosis, senile systemic amyloidosis, pituitary gland amyloidosis, hereditary renal amyloidosis, and familial non-neuropathic amyloidosis.

In some related aspects, the protein misfolding and deposition disease comprises an amyloid-associated disease. In one aspect, the amyloid-associated disease comprises Alzheimer's disease (AD), early onset Alzheimer's disease, late onset Alzheimer's disease, pre-symptomatic Alzheimer's disease or any combination thereof. In one aspect, the amyloid-associated disease is Alzheimer's disease (AD).

In some related aspects, the protein misfolding and deposition disease is selected from, but not restricted to, Parkinson's disease and related synucleinopathies, Huntington's disease, prion disease, mad cow disease, Creutzfeldt-Jacob disease, spongioform encephalopathy, frontotemporal dementia spectrum disorders, amyotrophic lateral sclerosis, familial British and Danish dementia, Finnish hereditary amyloidosis, familial non-neuropathic amyloidosis, and retinitis pigmentosa.

In some related aspects, the administration of Compound (1) is systemic, using oral administration, rectal administration, transmucosal administration, intranasal administration, intramuscular administration, subcutaneous administration, percutaneous administration, intrathecal administration, direct intracerebroventricular administration, intravenous administration, intraperitoneal administration or intranasal instillation.

In some related aspects, the administration is an oral administration.

In some further related aspects, the oral administration comprises a dose in the range of 0.03-0.3 mg/kg of compound (1).

In some further related aspects, the intravenous administration comprises a dose in the range of 0.01-0.1 mg/kg of compound (1).

In some further aspects, the amyloid-associated disease is selected from neurodegenerative retina diseases, particularly glaucoma or age-related macular degeneration (AMD). In some aspects, glaucoma is selected from the group consisting of primary angle-closure glaucoma, open-angle glaucoma (both primary and secondary), wide-angle glaucoma, steroid-induced glaucoma, traumatic glaucoma, secondary angle-closure glaucoma, and neovascular glaucoma. In some aspects, the age-related macular degeneration (AMD) is selected form the group consisting of dry and wet form of AMD, also including geographic atrophy (GA) secondary to AMD.

In some further aspects, the amyloid-associated disease is selected from diabetic retinopathy. In some further aspects, the diabetic retinopathy is non-proliferative type diabetic retinopathy proliferative type of diabetic retinopathy or combination thereof. In some aspects, the administration is an ocular administration.

In some aspects, the ocular administration is by eyedrops, eye creams, eye ointments, eye sprays, intraocular depot formulations, by injection including intraocular injection, intra- or periocular injections, comprising intraocular fluids, depot formulations, solid and semisolid intraocular carriers and matrices, or ocular devices, like contact lenses and films.

In some aspects, the topical ocular administration comprises eyedrops containing less than 1 mg of compound (1) per drop. In some further aspects, the ocular administration comprises a dose of less than 3 mg of compound (1) per drop.

In some aspects, the method comprises administering to said subject an initial loading dose of compound (1) and further administering multiple subsequent maintenance doses of the compound. In some aspects, the subsequent maintenance doses are lower doses of compound (1).

In some aspects, the compound is administered to the subject daily for a first period of at least one day, followed by a second period of at least one week wherein the compound is not administered, followed by repeating said first period of at least one day wherein said compound is administered to the subject daily.

In some aspects, the compound (1) is an active ingredient of a pharmaceutical composition which also includes a physiologically acceptable carrier. In some further aspects, the pharmaceutical composition is suitable for oral administration. In some further aspects, the pharmaceutical composition is suitable for ocular administration.

In some aspects, disclosed herein is a method of inhibiting amyloid beta Aβ toxicity in a subject, comprising administering low dose amount of compound (1)

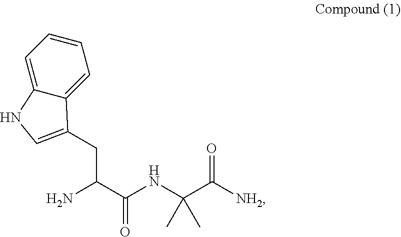 [SMILES: CC(C)(NC(=O)C(N)Cc1c[nH]c2ccccc12)C(N)=O]

thereby inhibiting Aβ₁₋₄₂ toxicity in said subject.

In some aspects, disclosed herein is a method of reversing or preventing the toxic effect of the misfolded and aggregated protein amyloid beta, namely toxic Aβ oligomers, by using compound (1) in a relative undersupply (reversed stoichiometric ratio) to achieve stronger detoxifying effects.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 Represents a graphical illustration of compound (1)'s target affinity to amyloid beta Aβ₁₋₄₂ monomers using Surface Plasmon Resonance (SPR). Synthetic Aβ₁₋₄₂ was used for this binding assay performed with a Biacore X100 biosensor instrument equipped with two flow cells. Aβ₁₋₄₂ was covalently coupled to one flow cell of CM7 sensor chips using an amine coupling kit. HBS-EP was used as assay running buffer. The analyte compound (1) was injected over the sensor chip in concentrations ranging from 0.3 nM up to 1000 nM at a flow rate of 10 μL/min for 180 sec at 25° C. Responses were evaluated at steady state, plot against concentration and fit by the four-parameter logistic equation to a K_{D}=2.5 nM (n=4). The K_{D} (dissociation constant) is the measure for the target affinity and expresses the concentration of compound (1) that elicited one-half of the maximum binding.

FIG. 2 Represents a graphical illustration of compound (1)'s pharmacokinetics in the rat brain. Compound (1) was administered as subcutaneous (s.c.) injection at doses of 0.4 mg/kg (∇) and 2.0 kg/kg (O, Δ). A microdialysis probe has been placed in the prefrontal cortex of these adult male Sprague Dawley rats to measure the concentration of compound (1) in the brain interstitial fluid (ISF) using a HPLC system with MS/MS detector. The dialysate samples were collected at a flow rate of 0.10 μL/min with a carrier flow rate of 0.8 μL/min. The animals were either anesthetized or awake and freely moving. Microdialysis data over a period of 360 min are depicted on a linear scale as means of 4 measurements±SEM.

FIG. 3 Represents a graphical illustration of compound (1)'s dose-dependent effects on in vivo LTP in anesthetized rats at very low concentrations. Given are normalized LTP of population spike (PS) amplitude at 80-90 min post theta burst stimulation (TBS). Compound (1) has been administered s.c. at dose of 0.08 mg/kg, 0.4 mg/kg, and 2.0 mg/kg. Amyloid beta Aβ₁₋₄₂ has been injected into brain ventricles at a concentration that strongly suppresses LTP (black column). The s.c. dose of 2 mg/kg compound (1) (dark grey) normalizes the LTP signal as expected while reaching a brain concentration of ca. 60 nM (see FIG. 2). However, the presumed inactive dose of only 0.4 mg/kg (middle gray), resulting in a peak brain concentration of 10 nM (see FIG. 2), showed at least as potent efficacy on LTP. Reducing the dose by another factor 5 (light grey) lead to a loss of the pharmacological effect.

FIGS. 4A-B. FIG. 4A Illustrates plots from in vitro LTP experiments using rat brain slices that were superfused at 23° C. with 50 nM Aβ₁₋₄₂ and/or with 10 nM compound (1). The compound (1) solution was added for 90 min before HFS was delivered in the first input (dark grey circles). At that concentration compound (1) allowed the induction of LTP. To ensure the validity of the Aβ₁₋₄₂-mediated effect, interleaved experiments with Aβ₁₋₄₂ (50 nM, closed circles, n=12) were additionally performed in separate slices. In the presence of Aβ₁₋₄₂, compound (1) at 10 nM was able to prevent LTP blockade when LTP was induced in the second input (light grey circles). Representative field excitatory postsynaptic potentials (TEPSPs) are shown on top.

FIG. 4B Represents a graphical illustration of compound (1)'s dose-dependent effects on in vitro LTP in rat brain slices based on LTP experiments shown in FIG. 4A. Toxic Aβ₁₋₄₂ oligomers formed from 50 nM amyloid beta Aβ₁₋₄₂ monomers block the LTP response almost entirely (black column). 500 nM and 100 nM of compound (1) can only partially alleviate the toxic effect of Aβ₁₋₄₂. However, the 10-fold lower concentration of 10 nM has a significantly stronger effect and blocks Aβ₁₋₄₂ toxicity almost completely. Surprisingly, efficacy at 10 nM means that 1 molecule of compound (1) can detoxify 5 molecules of Aβ₁₋₄₂. This refutes the previous scientific status, which called for an excess of the detoxifying agent. Data are shown as means of 5 experiments (n=5)±standard deviations.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

A skilled artisan would appreciate that the term “comprising” encompasses inclusion of the recited elements, but not excluding others which may be optional.

A skilled artisan would understand that when a range of values is expressed, another embodiment includes one limit value and/or the other limit value of the range. Additionally, all ranges are inclusive and combinable.

### Method for Treatment

The inventors surprisingly found that, in contrast to the known assumption that there is a need for several molecules of amyloid aggregation inhibitors for the inhibition of one amyloid beta molecule, the stoichiometry is exactly the opposite in the case of compound (1). For the old compound GAL-101 (MRZ-99030), which is the structurally related and previous best in class amyloid beta aggregation inhibitor, it was found that at least ten GAL-101 molecules are needed for the inhibition of one amyloid beta molecule (stoichiometric ration 10:1). For other similar compounds like ALZ-801 and PRI-002, several hundred or thousand molecules of the detoxifying compound are needed for the inhibition of the toxicity caused by one amyloid beta molecule (stoichiometric ratio in the range of approximately 100:1 to 5000:1). The inventors of the present disclosure found that one molecule of compound (1) can inhibit ten (!) amyloid beta molecules, and even more (stoichiometric ratio 1:10). The clinical meaning of this surprising and amazing phenomenon is that the dose of compound (1) can be reduced by 90% and even by 99%—to achieve the desired clinical effect and as summarized in Table 1 that the detoxifying effect of compound (1) in 10-fold undersupply (stoichiometric ratio 1:10) is even more than double as strong compared to application in stoichiometric excess.

In some embodiments, disclosed herein is a method of treating or preventing a protein misfolding and deposition disease in a subject in need thereof, the method comprising administering to the subject a low dose low dose amount of compound (1)

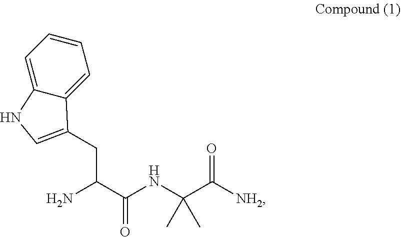 [SMILES: CC(C)(NC(=O)C(N)Cc1c[nH]c2ccccc12)C(N)=O]

thereby treating or preventing the protein misfolding and deposition disease in said subject.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

In one embodiment, “preventing” may encompass, inter alia, to delaying the onset of symptoms, preventing relapse to a disease, decreasing the number or frequency of relapse episodes, increasing latency between symptomatic episodes, or a combination thereof.

A skilled artisan would appreciate that “protein misfolding and deposition disease” arise when one of an ever-growing list of proteins (the amyloid-forming protein in its native conformation, also referred to as the amyloidogenic precursor) acquires an alternative folding state (the misfolded state), starts to aggregate and to form oligomers, then protofibrils and finally fibrillar structures (termed amyloid fibrils) which eventually deposit within tissues. Protein misfolding is believed to be the first step in the pathology of Alzheimer's disease, Creutzfeldt-Jakob disease, and other degenerative and neurodegenerative disorders.

In some embodiments, disclosed herein is a method of inhibiting Aβ toxicity in a subject, comprising administering low dose amount of compound (1)

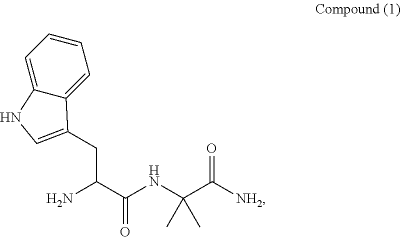 [SMILES: CC(C)(NC(=O)C(N)Cc1c[nH]c2ccccc12)C(N)=O]

thereby inhibiting Aβ toxicity in said subject.

In one embodiment, the low dose administration resulting in a highly efficient undersupply ratio between compound (1) and amyloid beta in the tissue leads to a concentration of less than 50 nM of compound (1) at the site of action, typically in the brain or in the retina. In another embodiment, the low dose administration results in a concentration of less than 40 nM of compound (1) at the site of action. In another embodiment, the low dose administration results in a concentration of less than 30 nM of compound (1) at the site of action. In another embodiment, the low dose administration results in a concentration of less than 20 nM of compound (1) at the site of action. In another embodiment, the low dose administration results in a concentration of less than 10 nM of compound (1) at the site of action. In another embodiment, the low dose administration results in a concentration of less than 5 nM of compound (1) at the site of action. In another embodiment, the low dose administration results in a concentration of less than 5 nM of compound (1) at the site of action. In another embodiment, the low dose administration results in a concentration of less than 3 nM of compound (1) at the site of action. In another embodiment, the low dose administration results in a concentration of less than 1 nM of compound (1) at the site of action.

In one embodiment, the low dose administration results in a concentration of 50 nM of compound (1) at the site of action. In another embodiment, the low dose administration results in a concentration of 40 nM of compound (1) at the site of action. In another embodiment, the low dose administration results in a concentration of 30 nM of compound (1) at the site of action. In another embodiment, the low dose administration results in a concentration of 20 nM of compound (1) at the site of action. In another embodiment, the low dose administration results in a concentration of 10 nM of compound (1) at the site of action. In another embodiment, the low dose administration results in a concentration of 5 nM of compound (1) at the site of action. In another embodiment, the low dose administration results in a concentration of 5 nM of compound (1) at the site of action. In another embodiment, the low dose administration results in a concentration of 3 nM of compound (1) at the site of action. In another embodiment, the low dose administration results in a concentration of 1 nM of compound (1) at the site of action.

A person skilled in the art would understand the term “site of action” as the site in the human body, where the pathology of the disease takes place. In one embodiment, wherein the site of action of the disease is the brain, the active concentration of compound (1) in the brain can be determined in cerebrospinal fluid (CSF) obtained from humans via lumbar puncture, for instance during a clinical Phase 1 dose-finding study. The targeted oral dose of compound (1) is that one corresponding to a CSF concentration of 10 nM.

In some embodiments, the administration is an oral administration.

In some embodiments, wherein the administration is oral administration, the low dose comprises 10 mg or less of compound (1). In one embodiment, wherein the administration is oral administration, the low dose comprises 10 mg of compound (1). In another embodiment, wherein the administration is oral administration, the low dose comprises 9.0 mg of compound (1). In another embodiment, wherein the administration is oral administration, the low dose comprises 8.0 mg of compound (1). In another embodiment, wherein the administration is oral administration, the low dose comprises 7.0 mg of compound (1). In another embodiment, wherein the administration is oral administration, the low dose comprises 6.0 mg of compound (1). In another embodiment, wherein the administration is oral administration, the low dose comprises 5.0 mg of compound (1). In another embodiment, wherein the administration is oral administration, the low dose comprises 4.0 mg of compound (1). In another embodiment, wherein the administration is oral administration, the low dose comprises 3.0 mg of compound (1).

In some embodiments, the administration is an intra vascular (IV) administration.

In some embodiments, wherein the administration is an IV administration, the low dose comprises 3 mg or less of compound (1). In one embodiment, wherein the administration is an IV administration, the low dose comprises 3 mg of compound (1). In another embodiment wherein the administration is an IV administration, the low dose comprises 2.5 mg of compound (1). In another embodiment wherein the administration is an IV administration, the low dose comprises 2.0 mg of compound (1). In another embodiment wherein the administration is an IV administration, the low dose comprises 1.5 mg of compound (1). In another embodiment wherein the administration is an IV administration, the low dose comprises 1.0 mg of compound (1).

In some embodiments, disclosed herein is a method of reversing or preventing the toxic effect of the misfolded and aggregated protein amyloid beta, namely toxic AB oligomers, by using compound (1) in a relative undersupply (reversed stoichiometric ratio) to achieve stronger detoxifying effects.

### Compound (1)

Compound (1), known as GAL-201 (formerly also known as MRZ-14042) is a proprietary small molecule with the following IUPAC nomenclature: (2R)-2-amino-N-(1-carbamomyl-1-methlyethyl)-3-(1H-indol-3-yl) propanamide. The molecular formula of the free base is C₁₅H₂₀N₄O₂ and the molecular weight is 288.34 g/Mol.

Compound (1) is represented by the formula below

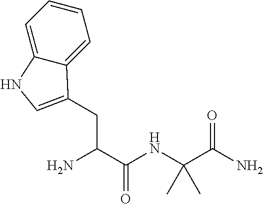 [SMILES: CC(C)(NC(=O)C(N)Cc1c[nH]c2ccccc12)C(N)=O]

Compound (1) is structurally related to GAL-101, the previous best in class compound. GAL-101 is 2-[2-Amino-3-(1H-indol-3-yl)-propionylamino]-2-methyl-propionic acid, C₁₅H₁₉N₃O₃, 289.33 g/Mol. It has a binding affinity to 28.9±2.9 nM to amyloid beta Aβ₁₋₄₂.

### Protein Misfolding and Deposition Disease

In some embodiments the present disclosure relates to a method of treating or preventing a protein misfolding and deposition disease in a subject in need thereof. In some embodiments, the protein misfolding and deposition disease is related to misfolding and deposition of Amyloid beta. In some embodiment, Amyloid beta comprises Aβ₁₋₄₂, Aβ₁₋₄₀, Aβₚ₃₋₄₂, derivates thereof or any combination thereof.

In some embodiments, the protein misfolding and deposition disease is selected from diseases associated with amyloid beta pathology, namely Alzheimer's disease (AD), early onset Alzheimer's disease, late onset Alzheimer's disease, pre-symptomatic Alzheimer's disease, type II diabetes mellitus, serum amyloid A disease (SAA amyloidosis), hereditary Icelandic syndrome, multiple myeloma, medullary carcinoma, aortic amyloidosis, cardiac amyloidosis, insulin injection amyloidosis, prion-systematic amyloidosis, chronic inflammation amyloidosis, senile systemic amyloidosis, pituitary gland amyloidosis, hereditary renal amyloidosis, and familial non-neuropathic amyloidosis.

In one embodiment, the protein misfolding and deposition disease comprises Alzheimer's disease (AD). In another embodiment, the protein misfolding and deposition disease comprises early onset Alzheimer's disease. In another embodiment, the protein misfolding and deposition disease comprises late onset Alzheimer's disease. In another embodiment, the protein misfolding and deposition disease comprises presymptomatic Alzheimer's disease. In another embodiment, the protein misfolding and deposition disease comprises type II diabetes mellitus. In another embodiment, the protein misfolding and deposition disease comprises serum amyloid A disease (SAA amyloidosis). In another embodiment, the protein misfolding and deposition disease comprises hereditary Icelandic syndrome. In another embodiment, the protein misfolding and deposition disease comprises insulin injection amyloidosis.

In another embodiment, the protein misfolding and deposition disease comprises multiple myeloma. In another embodiment, the protein misfolding and deposition disease comprises medullary carcinoma. In another embodiment, the protein misfolding and deposition disease comprises aortic amyloidosis. In another embodiment, the protein misfolding and deposition disease comprises cardiac amyloidosis. In another embodiment, the protein misfolding and deposition disease comprises Insulin injection amyloidosis. In another embodiment, the protein misfolding and deposition disease comprises prion-systematic amyloidosis. In another embodiment, the protein misfolding and deposition disease comprises chronic inflammation amyloidosis. In another embodiment, the protein misfolding and deposition disease comprises senile systemic amyloidosis. In another embodiment, the protein misfolding and deposition disease comprises pituitary gland amyloidosis.

In another embodiment, the protein misfolding and deposition disease comprises hereditary renal amyloidosis. In another embodiment, the protein misfolding and deposition disease comprises familial non-neuropathic amyloidosis.

In another embodiment, the protein misfolding and deposition disease comprises Parkinson disease, Huntington's disease, Jacob-Creutzfeldt disease, prion disease or mad cow disease. In another embodiment, the protein misfolding and deposition disease comprises Parkinson disease. In another embodiment, the protein misfolding and deposition disease comprises Huntington's disease. In another embodiment, the protein misfolding and deposition disease comprises Jacob-Creutzfeldt disease. In another embodiment, the protein misfolding and deposition disease comprises prion disease. In another embodiment, the protein misfolding and deposition disease comprises mad cow disease.

In another embodiment, the protein misfolding and deposition disease comprises amyloid-related ocular diseases and disorders. In another embodiment, the protein misfolding and deposition disease comprises prion disease.

In another embodiment, the protein misfolding and deposition disease comprises an amyloid-associated disease.

A skilled artisan would understand “amyloid-associated disease” as a pathology that is characterized by an accumulation and deposition of amyloid proteins in certain tissues of the body accompanied by a functional deficit of the respective organs.

In one embodiment, wherein the amyloid-associated disease comprises Alzheimer's disease (AD), early onset Alzheimer's disease, late onset Alzheimer's disease, pre-symptomatic Alzheimer's disease or any combination thereof, the treating or preventing comprises improvement of cognitive deficiencies, improvement of memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject. In another embodiment, the treating or preventing comprises improvement of cognitive deficiencies. In another embodiment, the treating or preventing comprises improvement of memory loss. In another embodiment, the treating or preventing comprises reduction of abnormal behavior. In another embodiment, the treating or preventing comprises reduction of hallucinations. In another embodiment, the treating or preventing comprises reduction of loss of spatial orientation. In another embodiment, the treating or preventing comprises reduction of apraxia. In another embodiment, the treating or preventing comprises reduction of aggression. In another embodiment, the treating or preventing comprises improvement in the ability to perform activities of daily living. In another embodiment, the treating or preventing comprises improvement in other symptoms of dementia.

In another embodiment, the amyloid-associated disease comprises glaucoma or age-related macular degeneration (AMD). In another embodiment, the amyloid-associated disease comprises glaucoma. In another embodiment, the amyloid-associated disease comprises age-related macular degeneration (AMD). In another embodiment, the amyloid-associated disease comprises geographic atrophy (GA) secondary to AMD.

A skilled artisan would understand glaucoma as a group of eye diseases that result in damage to the optic nerve (or retina) and cause vision loss. Risk factors for glaucoma include increasing age, high pressure in the eye, a family history of glaucoma, and use of steroid medication.

In one embodiment, the glaucoma is selected from the group consisting of primary angle-closure glaucoma, open-angle glaucoma (both primary and secondary), wide-angle glaucoma, steroid-induced glaucoma, traumatic glaucoma, secondary angle-closure glaucoma, and neovascular glaucoma.

In another embodiment, the glaucoma comprises primary angle-closure glaucoma. In another embodiment, the glaucoma comprises secondary open-angle glaucoma. In another embodiment, the glaucoma comprises primary open-angle glaucoma. In another embodiment, the glaucoma comprises wide-angle glaucoma. In another embodiment, the glaucoma comprises steroid-induced glaucoma. In another embodiment, the glaucoma comprises traumatic glaucoma. In another embodiment, the glaucoma comprises secondary angle-closure glaucoma. In another embodiment, the glaucoma comprises neovascular glaucoma.

As skilled artisan would understand age-related macular degeneration (AMD) is a medical condition which may result in blurred or no vision in the center of the visual field. Early on there are often no symptoms. Over time, however, some people experience a gradual worsening of vision that may affect one or both eyes. While it does not result in complete blindness, loss of central vision can make it hard to recognize faces, drive, read, or perform other activities of daily life. Visual hallucinations may also occur, but these do not represent a mental illness.

In one embodiment, the AMD is selected from Dry form and Wet form. In another embodiment, the AMD is dry form. In another embodiment, the AMD is wet form. A skilled artisan would appreciate that the difference between these two forms is the change of macula. People with dry form AMD can have drusen in their macula which typically grow over time and are associated with damage of light-sensitive cells (photoreceptors and the supporting pigment epithelium cells) and loss of vision. While people with wet form AMD, blood vessels sprout underneath their macula which leads to the leakage of blood and fluid into their retina.

In one embodiment, the amyloid-associated disease is diabetic retinopathy. In another embodiment, the diabetic retinopathy comprises non-proliferative type of diabetic retinopathy, proliferative type of diabetic retinopathy or combination thereof.

In one embodiment, the protein misfolding and deposition disease is diabetic retinopathy, which can range from non-proliferative diabetic retinopathy and its stages to proliferative diabetic retinopathy. As the disease progresses, associated diabetic macular edema may also become apparent. This disease of the retina can be associated with the deposition of amyloid beta or not.

### Dosage and Administration

In some embodiments, the administration is systemic administration.

In one embodiment, the systemic administering is by oral administration, rectal administration, transmucosal administration, transnasal administration, intramuscular administration, subcutaneous administration, intrathecal administration, direct intracerebroventricular administration, intravenous administration, intraperitoneal administration or intranasal instillation.

In another embodiment, the systemic administering is by oral administration. In another embodiment, the systemic administering is by rectal administration. In another embodiment, the systemic administering is by transmucosal administration. In another embodiment, the systemic administering is by transnasal administration. In another embodiment, the systemic administering is by intramuscular administration. In another embodiment, the systemic administering is by subcutaneous administration. In another embodiment, the systemic administering is by intrathecal administration. In another embodiment, the systemic administering is by direct intracerebroventricular administration.

In another embodiment, the systemic administering is by intravenous administration. In another embodiment, the systemic administering is by intraperitoneal administration. In another embodiment, the systemic administering is by intranasal instillation.

In one embodiment, the oral administration comprises a dose in the range of 0.03-0.3 mg/kg of compound (1). In another embodiment, the oral administration comprises a dose of 0.03 mg/kg of compound (1). In another embodiment, the oral administration comprises a dose of 0.04 mg/kg of compound (1). In another embodiment, the oral administration comprises a dose of 0.05 mg/kg of compound (1). In another embodiment, the oral administration comprises a dose of 0.06 mg/kg of compound (1). In another embodiment, the oral administration comprises a dose of 0.07 mg/kg of compound (1). In another embodiment, the oral administration comprises a dose of 0.08 mg/kg of compound (1). In another embodiment, the oral administration comprises a dose of 0.09 mg/kg of compound (1). In another embodiment, the oral administration comprises a dose of 0.1 mg/kg of compound (1). In another embodiment, the oral administration comprises a dose of 0.12 mg/kg of compound (1). In another embodiment, the oral administration comprises a dose of 0.13 mg/kg of compound (1). In another embodiment, the oral administration comprises a dose of 0.14 mg/kg of compound (1). In another embodiment, the oral administration comprises a dose of 0.15 mg/kg of compound (1). In another embodiment, the oral administration comprises a dose of 0.16 mg/kg of compound (1). In another embodiment, the oral administration comprises a dose of 0.17 mg/kg of compound (1). In another embodiment, the oral administration comprises a dose of 0.18 mg/kg of compound (1). In another embodiment, the oral administration comprises a dose of 0.19 mg/kg of compound (1). In another embodiment, the oral administration comprises a dose of 0.2 mg/kg of compound (1). In another embodiment, the oral administration comprises a dose of 0.21 mg/kg of compound (1). In another embodiment, the oral administration comprises a dose of 0.22 mg/kg of compound (1). In another embodiment, the oral administration comprises a dose of 0.23 mg/kg of compound (1). In another embodiment, the oral administration comprises a dose of 0.24 mg/kg of compound (1). In another embodiment, the oral administration comprises a dose of 0.25 mg/kg of compound (1). In another embodiment, the oral administration comprises a dose of 0.26 mg/kg of compound (1). In another embodiment, the oral administration comprises a dose of 0.27 mg/kg of compound (1). In another embodiment, the oral administration comprises a dose of 0.28 mg/kg of compound (1). In another embodiment, the oral administration comprises a dose of 0.29 mg/kg of compound (1). In another embodiment, the oral administration comprises a dose of 0.3 mg/kg of compound (1).

In one embodiment, the intravenous administration comprises a dose in the range of 0.01-0.1 mg/kg of compound (1). In one embodiment, the intravenous administration comprises a of 0.01 mg/kg of compound (1). In another embodiment, the intravenous administration comprises a dose of 0.02 mg/kg of compound (1). In another embodiment, the intravenous administration comprises a dose of 0.03 mg/kg of compound (1). In another embodiment, the intravenous administration comprises a dose of 0.04 mg/kg of compound (1). In another embodiment, the intravenous administration comprises a dose of 0.05 mg/kg of compound (1). In another embodiment, the intravenous administration comprises a dose of 0.06 mg/kg of compound (1). In another embodiment, the intravenous administration comprises a dose of 0.07 mg/kg of compound (1). In another embodiment, the intravenous administration comprises a dose of 0.08 mg/kg of compound (1). In another embodiment, the intravenous administration comprises a dose of 0.09 mg/kg of compound (1). In another embodiment, the intravenous administration comprises a dose of 0.1 mg/kg of compound (1).

In some embodiments, the administration is an ocular administration.

In one embodiment, the ocular administration is by eye drops, eye creams, eye ointments, eye sprays, intraocular depot formulations, by injection including intraocular injection, intra- or periocular injections, comprising intraocular fluids, depot formulations, solid and semisolid intraocular carriers and matrices, or ocular devices, like contact lenses and films. In another embodiment, the ocular administration is by eye drops. In another embodiment, the ocular administration is by eye creams. In another embodiment, the ocular administration is by eye ointments. In another embodiment, the ocular administration is by eye sprays. In another embodiment, the ocular administration is by intraocular depot formulations. In another embodiment, the ocular administration is by injection including intraocular injection, intra- or periocular injections, comprising intraocular fluids, depot formulations, solid and semisolid intraocular carriers and matrices. In another embodiment, the ocular administration is by ocular devices, like contact lenses and films.

In one embodiment, the ocular administration comprises a single dose less than 1 mg of compound (1) per eye. In another embodiment, the ocular administration comprises a single dose less than 0.8 mg of compound (1) per eye. In another embodiment, the ocular administration comprises a single dose less than 0.6 mg of compound (1). In another embodiment, the ocular administration comprises a single dose less than 0.4 mg of compound (1). In another embodiment, the ocular administration comprises a single dose less than 0.2 mg of compound (1). The abovementioned single doses are typically repeated once to three times daily over long treatment periods up to live long treatment.

In one embodiment, the method comprises administering to said subject an initial loading dose and further administering multiple subsequent maintenance doses of compound (1). In one embodiment, the subsequent maintenance doses are lower doses of compound (1).

In one embodiment, the initial loading dose is administered 1-3 times per day for 1 day-1 month.

In another embodiment, the initial loading dose is administered 1 time per day for 1 day-1 month. In another embodiment, the initial loading dose is administered 2 times per day for 1 day-1 month. In another embodiment, the initial loading dose is administered 3 times per day for 1 day-1 month.

In another embodiment, the initial loading dose is administered 1-3 times per day for 1 day. In another embodiment, the initial loading dose is administered 1-3 times per day for 5 days. In another embodiment, the initial loading dose is administered 1-3 times per day for 10 days. In another embodiment, the initial loading dose is administered 1-3 times per day for 15 days. In another embodiment, the initial loading dose is administered 1-3 times per day for 20 days. In another embodiment, the initial loading dose is administered 1-3 times per day for 25 days. In another embodiment, the initial loading dose is administered 1-3 times per day for 30 days.

In one embodiment, the maintenance dose is administered once a week, once every two weeks or once every four weeks. In another embodiment, the maintenance dose is administered once a week. In another embodiment, the maintenance dose is administered once every two weeks. In another embodiment, the maintenance dose is administered once every four weeks.

In one embodiment, the maintenance dose is 10%-75% of the initial loading dose. In another embodiment, the maintenance dose is 10% of the initial loading dose. In another embodiment, the maintenance dose is 20% of the initial loading dose. In another embodiment, the maintenance dose is 30% of the initial loading dose. In another embodiment, the maintenance dose is 40% of the initial loading dose. In another embodiment, the maintenance dose is 50% of the initial loading dose. In another embodiment, the maintenance dose is 60% of the initial loading dose. In another embodiment, the maintenance dose is 70% of the initial loading dose. In another embodiment, the maintenance dose is 75% of the initial loading dose.

In one embodiment, the compound is administered to the subject daily for a first period of at least one day, followed by a second period of at least one week wherein the compound is not administered, followed by repeating the first period of at least one day wherein the compound is administered to the subject daily.

### Pharmaceutical Composition

The herein-described compositions can be incorporated into pharmaceutical compositions suitable for administration. In one embodiment, a “pharmaceutical composition” or a “pharmaceutical formulation” refers to a preparation of one or more of the active ingredients described herein with other chemical components such as physiologically suitable carriers and excipients. The purpose of a pharmaceutical composition or a “pharmaceutical formulation” is to facilitate administration of a compound to a subject. In certain embodiments, a “pharmaceutical composition” or a “pharmaceutical formulation” provides the pharmaceutical dosage form of a drug.

In some embodiments, “active ingredient” refers to the molecule which is accountable for the biological effect. In some embodiments, the active ingredient comprises compound (1).

In some embodiments, the pharmaceutical composition comprises an excipient. A skilled artisan would appreciate that, in some embodiments, excipients are selected according to the delivery mode of the pharmaceutical composition.

In one embodiment, the compound (1) is an active ingredient of a pharmaceutical composition which also includes a physiologically acceptable carrier.

Pharmaceutical compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol (PEG); alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of which has been adjusted to about 7.4.

In some embodiments, the pharmaceutical compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol (PEG), sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates), adjuvants, or any combination thereof.

In one embodiment, the pharmaceutical composition is suitable for oral administration, rectal administration, transmucosal administration, transnasal administration, intramuscular administration, subcutaneous administration, intrathecal administration, direct intracerebroventricular administration, intravenous administration, intraperitoneal administration, intranasal instillation, or ocular administrations.

In one embodiment, the pharmaceutical composition is suitable for oral administration. In another embodiment, the pharmaceutical composition is suitable for rectal administration. In another embodiment, the pharmaceutical composition is suitable for transmucosal administration. In another embodiment, the pharmaceutical composition is suitable for transnasal administration. In another embodiment, the pharmaceutical composition is suitable for intramuscular administration. In another embodiment, the pharmaceutical composition is suitable for subcutaneous administration. In another embodiment, the pharmaceutical composition is suitable for intrathecal administration. In another embodiment, the pharmaceutical composition is suitable for direct intracerebroventricular administration. In another embodiment, the pharmaceutical composition is suitable for intravenous administration. In another embodiment, the pharmaceutical composition is suitable for intraperitoneal administration. In another embodiment, the pharmaceutical composition is suitable for intranasal instillation. In another embodiment, the pharmaceutical composition is suitable for ocular administration.

In some embodiments, compositions including the preparation disclosed herein formulated in a compatible pharmaceutical carrier are also prepared, placed in an appropriate container, and labeled for treatment of an indicated condition.

A skilled artisan would appreciate that foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropanol. Application and agitation of such a composition at the body temperature cause the isopropanol to vaporize and generate the foam, in a manner similar to a pressurized aerosol foaming system. Foams can be water-based or hydroalcoholic, but are frequently formulated with high alcohol content which, upon application to the skin of a user, quickly evaporates, driving the active ingredient through the upper skin layers to the site of treatment.

In one embodiment, the formulations provided herein also comprise preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcysteine, sodium metabisulfite and others; aromatic agents; viscosity adjustors, such as polymers, including cellulose and derivatives thereof; and polyvinyl alcohol and acid and bases to adjust the pH of these aqueous compositions as needed. The compositions also comprise local anesthetics or other actives. The compositions can be used as sprays, mists, drops, and the like.

### EXAMPLES

### Example 1: Compound (1) Target Affinity to Amyloid Beta Aβ₁₋₄₂

The aim of this experiment was to evaluate the target affinity of compound (1) to misfolded Aβ₁₋₄₂ monomers. The affinity was determined using in vitro Surface Plasmon Resonance (SPR) technology. SPR experiments allow the investigation of binding of compounds to lower concentrations of Aβ₁₋₄₂ and offer the possibility to directly asses the affinity of such binding. A Biacore X100 SPR instrument, equipped with two flow cells on a sensor chip, was used for real-time binding studies.

Aβ₁₋₄₂ (American Peptide Company, Sunnyvale, CA, USA) was dissolved to 1 mg/ml in hexafluoroisopropanol (HFIP). The tube was incubated at room temperature for 1.5 hrs while shaking, 100 μg aliquots were prepared in low binding Eppendorf tubes and frozen at −80° C. for 30-60 min. After lyophilization over night the aliquots were stored at −20° C. until use. For the preparation of monomers, one HFIP treated Aβ aliquot was thawed and freshly dissolved in DMSO (anhydrous), This 5 mM stock solution was centrifuged (5 min 13000 g) and the supernatant diluted to 100 μM in 10 mM sodium acetate, pH 4.0 immediately before immobilization.

Aβ monomers were covalently coupled to the flow cells of the CM7 sensor chips (carboxymethylated dextran matrix attached to gold surface) with ca.1 pg/mm² Aβ₁₋₄₂ density on the surface matrix. For immobilization of human Aβ₁₋₄₂ monomers, HFIP-treated peptide was dissolved in DMSO to 5 mM, diluted to 100 μM in 10 mM sodium acetate pH 4.0 and immediately coupled to the surface of one flow cell of the sensor chip. The second flow cell was used as a reference and treated with ethanolamine instead of Aβ. To determine the affinity, compound (1) was tested in concentrations ranging from 0.3 nM up to 1000 nM using HBS-EP, 0.1% DMSO as a running buffer at 25° C.

The resonance units (RUs) elicited by the compound injected into the ethanolamine control flow cell was set as reference response and subtracted from the RUs elicited by the same compound injected to the Aβ saturated flow cell. The relationships between each RU obtained at the steady state of binding (plateau of the binding curve) and each concentration of the compound were plotted. Biacore X100 software Ver 1.1 was used to record the binding curves and to analyse them (plot each RU at the steady state vs concentration of analyte, fit the plot, determine K_{D} values). The dissociation equilibrium constant K_{D} of the analyte to the immobilized Aβ was determined from the steady-state levels estimating the maximum RU Rₘₐₓ and calculating the K_{D} as the concentration of the compound that elicited one-half of the Rₘₐₓ.

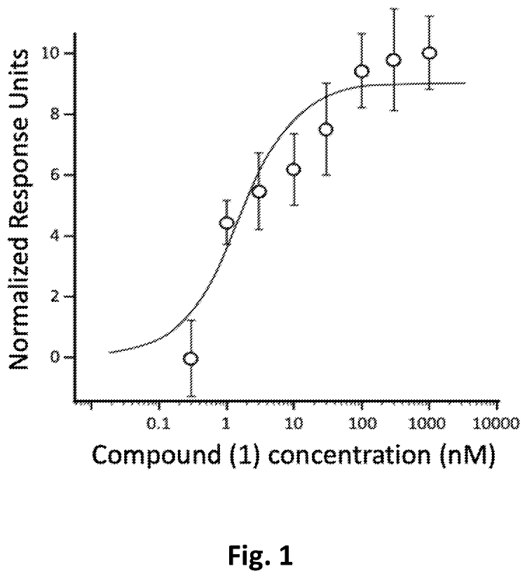

The Kp for compound (1) at Aβ monomers is 2.5±0.6 nM (n=4). The results are presented in FIG. 1. The affinity was 4-fold stronger to the target compared to previous best in class compound GAL-101.

### Example 2: Compound (1)'s Pharmacokinetics in the Rat Brain

The aim of this experiment was to evaluate the pharmacokinetics of compound (1) in the rat brain. Compound (1) was administered subcutaneously to anesthetized or awake and freely moving animals. A microdialysis probe has been placed in the prefrontal cortex of these adult male Sprague Dawley rats to measure the concentration of compound (1) in the brain interstitial fluid (ISF) using a HPLC system with MS/MS detector.

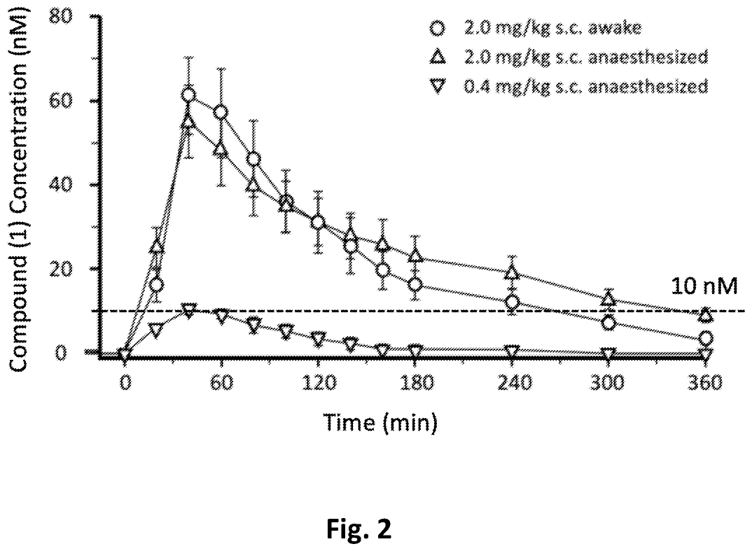

Results are presented in FIG. 2.

### Example 3: Compound (1)'s Dose Dependent Effects on In Vivo LTP in Anesthetized Rates at Very Low Concentrations

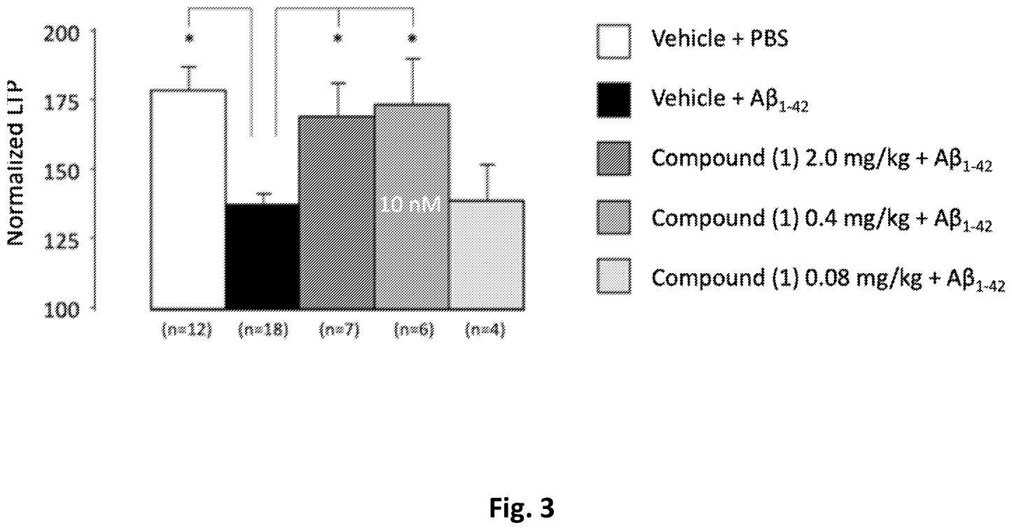

The aim of this experiment was to evaluate the dose-dependent effects of compound (1) on in vivo LTP in anesthetized rats at very low concentrations. Compound (1) has been administered s.c. at dose of 0.08 mg/kg, 0.4 mg/kg, and 2.0 mg/kg. Amyloid beta Aβ₁₋₄₂ has been injected into brain ventricles at a concentration that strongly suppresses LTP. As evident in FIG. 3, surprisingly, the presumed inactive dose of only 0.4 mg/kg (middle gray), resulting in a peak brain concentration of 10 nM, showed at least as potent efficacy on LTP.

### Example 4: Dose-Dependent Effects of Compound (1) on In Vitro LTP Experiments

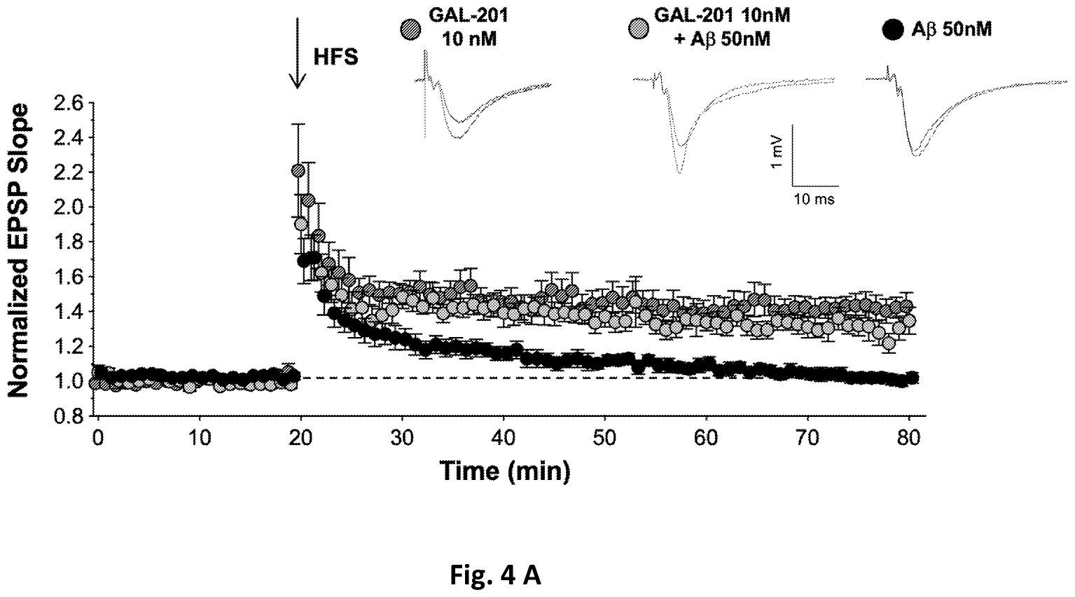

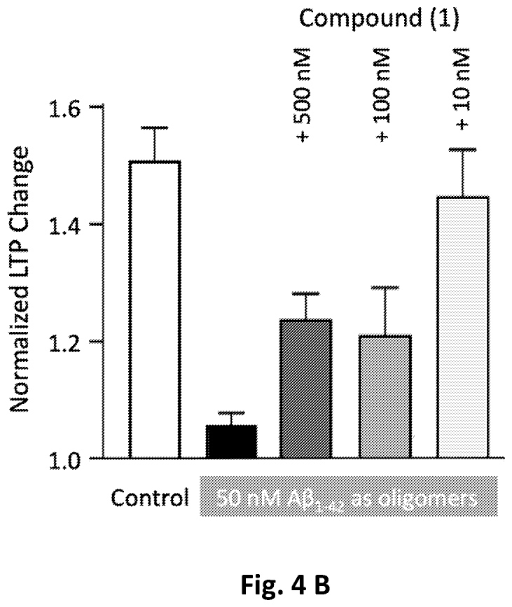

For the long-term potentiation (LTP) experiments brain slices from 2 months old mice were used. Transverse hippocampal slices (350 μm thick) were obtained after decapitation under isoflurane-anesthesia. All slices were placed in a holding chamber for at least 60 min and were then transferred to a super fusing chamber for extracellular recordings. The flow rate of the solution through the chamber was 4 ml/min. The composition of the solution was 124 mM NaCl, 3 mM KCl, 26 mM NaHCO₃, 2 mM CaCl₂), 1 mM MgSO₄, 10 mM D-glucose, and 1.25 mM NaH₂PO₄, bubbled with a 95% O₂/5% CO₂ mixture, and had a final pH of 7.3. All experiments were performed at room temperature. Aβ₁₋₄₂ stock solution in DMSO was added to the bath solution to give a final concentration of 50 nM. Extracellular recordings of field excitatory postsynaptic potentials (fEPSPs) were obtained from the dendritic region of the CA1 region of the hippocampus using glass micropipettes filled with superfusion solution. For all recordings both stimulating electrodes were used to utilize the input specificity of LTP and thereby allow the measurement of an internal control within the same slice. Steady baseline recordings were made for at least 30 minutes before application of tetanic stimuli. For LTP induction, high-frequency stimulation (HFS) conditioning pulses (100 Hz/1 s; 4-5 V) were applied to the Schaffer collateral commissural pathway via two independent inputs. Before inducing LTP, Aβ₁₋₄₂ has been applied for 90 min. HFS was delivered from one of the electrodes under conditions in the presence of Compound (1) and potentiation of the responses was monitored for at least 60 min after the tetanus. Either Aβ₁₋₄₂ was then applied via the bath solution for 90 min before attempting to induce LTP in the second input following HFS delivered via the second electrode. Amplified fEPSPs were filtered (3 kHz), digitized (15 kHz), measured and plotted (FIG. 4A). Measurements of the slope of the fEPSP were taken between 20% and 80% of the peak amplitude. Slopes of fEPSPs were normalized with respect to the 20 min control period before tetanic stimulation.

Compound (1) was used at various concentrations in these LTP experiments. Surprisingly, when compound (1) was applied at the lowest concentration of only 10 nM it best prevented the detrimental effect of 50 nM Aβ₁₋₄₂ on hippocampal LTP. FIG. 4B summarizes the effects of different compound (1) concentrations leading to different stoichiometric Aβ₁₋₄₂/compound (1) ratios (10:1, 2:1, 1:5). The LTP signals (fEPSP potentiation) calculated as the average of the last 50-60 mins after HFS are presented as a bar diagram.

The reversal of the stoichiometric ratio between compound (1) and amyloid beta Aβ₁₋₄₂ are presented in Table 1, below.

TABLE 1

| Concentration compound (1) | 500 nM | 100 nM | 10 nM |
|---|---|---|---|
| Stoichiometric ratio | 10:1 | 2:1 | [symbol] |
| compound (1):Aβ₁₋₄₂ | | | 1:5 |
| Relative effect size | 40.2% | 34.7% | 88.1% |
| (blocking Aβ₁₋₄₂ toxicity) | | | |

This table shows that the reversal of the stoichiometric ratio between compound (1) and amyloid beta Aβ₁₋₄₂ potentiates the detoxifying strength of compound (1). The data of this table are taken from the experiment presented in FIG. 4B. The control experiment is defined as 100% normalized LTP response and the toxic effect of 50 nM Aβ₁₋₄₂ oligomers is defined as 0% normalized LTP change. Using the usual stoichiometric excess for compound (1), namely 500 nM or 100 nM, leads to a weak detoxifying effect of 40.2% and 34.7%, respectively. However, reversing the stoichiometric ratio and using compound (1) at a very low concentration (10 nM) representing a 5-fold undersupply, significantly improves the detoxifying strength reaching 88.1%.

The control experiment is defined as 100% normalized LTP response and under the toxic effect of 50 nM Aβ₁₋₄₂ oligomers the normalized LTP change is defined as 0%. Using the usual stoichiometric excess for Compound (1), namely 500 nM or 100 nM, a moderate detoxifying effect of 40.2% and 34.7% has been observed, respectively. However, reversing the stoichiometric ratio and using compound (1) at a concentration of only 10 nM, representing a 5-fold stoichiometric undersupply, significantly improves the detoxifying strength reaching 88.1% effect size.

Combining the calculations from the in vitro and in vivo LTP experiments demonstrated that a subcutaneous (s.c.) dose of 0.4 mg/kg compound (1), which leads to a 10 nM concentration of compound (1) in the extracellular space of the brain, completely detoxified the present toxic Aβ₁₋₄₂ and lead to a fully restored LTP signal (see FIG. 3).

In animals subject to intracerebroventricular (i.c.v.) injection of 6 μL oligomeric Aβ₁₋₄₂ solution (1.67 mM) significant deficits in LTP were observed. (FIG. 3). At the end of the recording period, 80-90 min after LTP induction, the PS amplitude following i.c.v. administration of oligomeric Aβ₁₋₄₂ measured 138.1±4.1% of baseline (n=18), compared to 179.2±8.7% in PBS-injected animals (n=12). In animals subjected to i.c.v injection of 6 μL oligomeric Aβ₁₋₄₂, 3 different s.c. doses of compound (1), namely 0.08, 0.4 and 2 mg/kg, were administrated before i.c.v. injection of oligomeric Aβ₁₋₄₂. 0.4 mg/kg and 2 mg/kg compound (1) brought post-TBS responses towards those observed in control animals (P<0.05, one-way ANOVA with post-hoc Bonferroni test). At the end of the recording period, 80-90 min after LTP induction, the PS amplitude following s.c. administration of compound (1) and i.c.v. administration of oligomeric Aβ₁₋₄₂ measured 170.0±11.9% of baseline (n=7) for the 2 mg/kg dose, 174.7±16.0% of baseline (n=6) for the 0.4 mg/kg dose, and 139.6±13.1% of baseline (n=4) for the 0.08 mg/kg dose, compared to 179.2±8.7% in vehicle-treated controls (n=12) and 138.1±4.1% i.c.v. oligomeric Aβ₁₋₄₂-injected animals (n=18). These results confirm the high potency of compound (1) to suppress Aβ₁₋₄₂ toxicity found in the above-described in vitro experiments (presented in FIG. 4B).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating or preventing a protein misfolding and deposition disease in a subject in need thereof, the method comprising administering to the subject a low dose amount of compound (1) 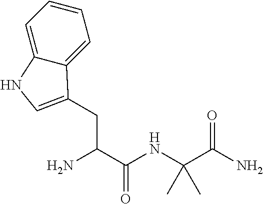 [SMILES: CC(C)(NC(=O)C(N)Cc1c[nH]c2ccccc12)C(N)=O]
Compound (1),
thereby treating or preventing the protein misfolding and deposition disease in said subject.

2. The method according to claim 1, wherein said low dose administration results in a concentration of compound (1) of less than 50 nM at the site of action.

3. (canceled)

4. (canceled)

5. (canceled)

6. The method according to claim 1, wherein said protein misfolding and deposition disease is selected from Alzheimer's disease (AD), early onset Alzheimer's disease, late onset Alzheimer's disease, pre-symptomatic Alzheimer's disease, type II diabetes mellitus, serum amyloid A disease (SAA amyloidosis), hereditary Icelandic syndrome, multiple myeloma, medullary carcinoma, aortic amyloidosis, cardiac amyloidosis, insulin injection amyloidosis, prion-systematic amyloidosis, chronic inflammation amyloidosis, senile systemic amyloidosis, pituitary gland amyloidosis, hereditary renal amyloidosis, familial non-neuropathic amyloidosis, Parkinson's disease, Huntington's disease, Jacob-Creutzfeld disease, prion disease, and mad cow disease.

7. (canceled)

8. The method according to claim 1, wherein said protein misfolding and deposition disease comprises an amyloid-associated disease.

9. The method according to claim 8, wherein said amyloid-associated disease comprises Alzheimer's disease (AD), early onset Alzheimer's disease, late onset Alzheimer's disease, pre-symptomatic Alzheimer's disease or any combination thereof.

10. The method according to claim 9, wherein said treating or preventing comprises improvement of cognitive deficiencies, improvement of memory loss, reduction of abnormal behavior, reduction of hallucinations, reduction of loss of spatial orientation, reduction of apraxia, reduction of aggression, improvement in the ability to perform activities of daily living, or other symptoms of dementia, or any combination thereof, in said subject.

11. The method according to claim 8, wherein said amyloid-associated disease comprises diabetic retinopathy.

12. The method according to claim 1, wherein said administration is a systemic administration.

13. The method according to claim 12, wherein said systemic administering is by oral administration, rectal administration, transmucosal administration, intranasal administration, intramuscular administration, subcutaneous administration, percutaneous administration, intrathecal administration, direct intracerebroventricular administration, intravenous administration, intraperitoneal administration or intranasal instillation.

14. The method according to claim 13, wherein said oral administration comprises a dose in the range of 0.03-0.3 mg/kg of compound (1).

15. The method according to claim 13, wherein said intravenous administration comprises a dose in the range of 0.01-0.1 mg/kg of compound (1).

16. The method according to claim 8, wherein said amyloid-associated disease is selected from glaucoma or age-related macular degeneration (AMD).

17. (canceled)

18. The method according to claim 16, wherein said administration is an ocular administration.

19. (canceled)

20. The method according to claim 18, comprising administering less than 1 mg of compound (1) as single dose to an eye of a subject in need.

21. The method according to claim 1, wherein the method comprises administering to said subject an initial loading dose and further administering multiple subsequent maintenance doses of said compound (1).

22. (canceled)

23. (canceled)

24. (canceled)

25. The method according to claim 1, wherein said compound is administered to the subject daily for a first period of at least one day, followed by a second period of at least one week wherein the compound is not administered, followed by repeating said first period of at least one day wherein said compound is administered to the subject daily.

26. The method of claim 1, wherein said compound (1) is an active ingredient of a pharmaceutical composition which also includes a physiologically acceptable carrier.

27. (canceled)

28. (canceled)

29. A method of inhibiting Aβ toxicity in a subject, comprising administering low dose amount of compound (1) 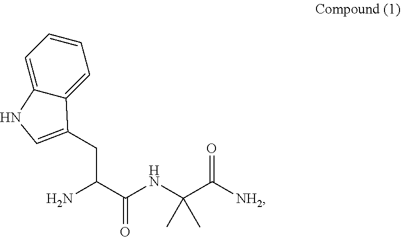 [SMILES: CC(C)(NC(=O)C(N)Cc1c[nH]c2ccccc12)C(N)=O]
thereby inhibiting Aβ toxicity in said subject.

30. The method according to claim 29, wherein said low dose administration results in a concentration of less than 50 nM at the site of action.

31. (canceled)

32. (canceled)

33. (canceled)

34. A method of reversing or preventing the toxic effect of the misfolded and aggregated protein amyloid beta, namely toxic Aβ oligomers, by using compound (1) in a relative undersupply (reversed stoichiometric ratio) to achieve stronger detoxifying effects.
